(19) United States
(12) Patent Application Publication
(10) Pub. No.: US 2024/0218336 A1
(43) Pub. Date: Jul. 4, 2024

(54) PRODUCTION OF RETINOL

(71) Applicant: DSM IP ASSETS B.V., Heerlen (NL)

(72) Inventors: Nathalie BALCH, Kaiseraugst (CH); Paul BLOMQUIST, Kaiseraugst (CH); Reed DOTEN, Kaiseraugst (CH); Peter HOUSTON, Kaiseraugst (CH); Ethan LAM, Kaiseraugst (CH); Jenna MCMAHON, Kaiseraugst (CH); Joshua TRUEHEART, Kaiseraugst (CH); Celine VIAROUGE, Kaiseraugst (CH)

(21) Appl. No.: 18/439,987

(22) Filed: Feb. 13, 2024

Related U.S. Application Data
(63) Continuation of application No. 16/648,851, filed on Mar. 19, 2020, now abandoned.
(60) Provisional application No. 62/562,699, filed on Sep. 25, 2017.
(60) Provisional application No. 62/562,712, filed on Sep. 25, 2017.
(60) Provisional application No. 62/562,612, filed on Sep. 25, 2017.

(51) Int. Cl. C12N 9/04; C12P 23/00

(52) U.S. Cl. CPC C12N 9/0006; C12P 23/00; C12Y 101/01105

(57) ABSTRACT

The present invention is related to a novel enzymatic process for production of vitamin A alcohol (retinol) via conversion of retinal, which process includes the use of heterologous enzymes having activity as retinal reductase, particularly wherein the reaction leads to at least about 90% conversion of retinal into retinol. Said process is particularly useful for biotechnological production of vitamin A.

### CROSS-REFERENCE TO RELATED APPLICATIONS

This application is a continuation of U.S. application Ser. No. 16/648,851, filed Mar. 19, 2020, which is the U.S. national phase of International Application No. PCT/EP2018/076031 filed Sep. 25, 2018, which designated the U.S. and claims priority to U.S. Provisional Patent Application Nos. 62/562,699 filed Sep. 25, 2017, 62/562,712 filed Sep. 25, 2017, and 62/562,612 filed Sep. 25, 2017, the entire contents of each of which are hereby incorporated by reference.

### REFERENCE TO A SEQUENCE LISTING SUBMITTED ELECTRONICALLY

The content of the electronically submitted sequence listing (Name: 4662_4670_Sequence_Listing.xml, Size: 16,382 bytes; and Date of Creation: Feb. 12, 2024) is herein incorporated by reference in its entirety.

The present invention is related to a novel enzymatic process for production of vitamin A alcohol (retinol) via conversion of retinal, which process includes the use of heterologous enzymes having activity as retinal reductase, particularly wherein the reaction leads to at least about 90% conversion of retinal into retinol. Said process is particularly useful for biotechnological production of vitamin A.

Retinol is an important intermediate/precursor in the process of retinoid production, particularly such as vitamin A production. Retinoids, including vitamin A, are one of very important and indispensable nutrient factors for human beings which have to be supplied via nutrition. Retinoids promote well-being of humans, inter alia in respect of vision, the immune system and growth.

Current chemical production methods for retinoids, including vitamin A and precursors thereof, have some undesirable characteristics such as e.g. high-energy consumption, complicated purification steps and/or undesirable by-products. Therefore, over the past decades, other approaches to manufacture retinoids, including vitamin A and precursors thereof, including microbial conversion steps, which would be more economical as well as ecological, have been investigated.

In general, the biological systems that produce retinoids are industrially intractable and/or produce the compounds at such low levels that commercial scale isolation is not practicable. There are several reasons for this, including instability of the retinoids in such biological systems or the relatively high production of by-products.

Thus, it is an ongoing task to improve the product-specificity and/or productivity of the enzymatic conversion of beta-carotene into vitamin A. Particularly, it is desirable to optimize the productivity of enzymes involved in conversion of retinal towards retinol, i.e. looking for enzymes with high retinal reducing activity.

Surprisingly, we now could identify specific retinol dehydrogenases (RDHs) which are capable of converting retinal into retinol with a total conversion of at least about 90% towards generation of retinol.

In particular, the present invention is directed to RDHs, preferably fungal RDHs which are heterologous expressed in a suitable host cell, such as a carotenoid-producing host cell, particularly a fungal host cell, with the activity of reducing retinal into retinol with a total conversion towards production of retinol of at least about 90%, preferably 92, 95, 97, 98, 99 or even 100% based on the total amount of retinoids produced by said host cell, i.e. an amount of retinol of at least about 90% compared to the amount of retinal present in said retinoid mix produced by the host cell.

The invention is in one aspect preferably directed to a carotenoid-producing host cell, in particular a retinoid-producing host cell, comprising such RDH as defined herein, said host cell producing a retinal mix comprising both retinol and retinal, wherein the percentage of retinol is at least about 90%, preferably 92, 95, 97, 98, 99 or even 100% based on the total amount of retinoids (comprising retinal/retinol) in the retinol mix.

The terms “retinal reductase”, “retinol dehydrogenase”, “enzyme having retinal reducing activity” or “RDH” are used interchangeably herein and refer to enzymes [EC 1.1.1.105] which are capable of catalyzing the conversion of retinal into retinol and also the backwards reaction leading to retinal, the latter activity is to be reduced to about 10% or less according to the present invention.

The terms “conversion”, “oxidation”, “reduction” in connection with enzymatic catalysis of retinol are used interchangeably herein and refer to the action of RDH as defined herein.

As used herein, the term “fungal host cell” includes particularly yeast as host cell, such as e.g. Yarrowia or Saccharomyces.

The RDHs as defined herein leading to total conversion of about at least 90% towards production of retinol from enzymatic catalysis of retinal are preferably introduced into a suitable host cell, i.e. expressed as heterologous enzymes, or might be expressed as endogenous enzymes. Preferably, the enzymes as described herein are expressed as heterologous enzymes.

For the purpose of the present invention, any retinal reducing enzyme which results in an increase of at least about 18%, such as e.g. at least about 20, 30, 40, 50, 60, 70, 80% towards formation of retinol can be used in a process as defined herein, such increase being calculated on the retinol formation using endogenous RDHs present in suitable carotenoid-producing host cells, particularly fungal host cells, such as e.g. strains of Yarrowia or Saccharomyces.

RDHs with activity towards retinol formation, i.e. retinal reduction reaction, as defined herein might be obtained from any source, such as e.g. plants, animals, including humans, algae, fungi, including yeast, or bacteria.

In one embodiment, the polypeptide having RDH activity as defined herein, i.e. with a total conversion of at least 90% towards retinol, are obtainable from fungi, in particular Dikarya or Mycoromycetes, including but not limited to fungi selected from Ascomycota or Mucorales, preferably obtained from Fusarium or Mucor, more preferably isolated from F. fujikuroi or M. circinelloides, such as a polypeptide with at least 60%, such as e.g. 65, 70, 75, 80, 85, 90, 95, 97, 98, 99% or up to 100% identity to F. fujikuroi FfRDH12 (polypeptide sequence derived from EXK27040) or M. circinelloides McRDH12 (polypeptide sequence derived from EPB85547.1), e.g. polypeptides with at least 60%, such as e.g. 65, 70, 75, 80, 85, 90, 95, 97, 98, 99% or up to 100% identity to a polypeptide according to SEQ ID NO: 1, including a polypeptide encoded by e.g. a polynucleotide according to SEQ ID NO:2.

In further embodiments, the polypeptide having RDH activity as defined herein, i.e. with a total conversion of at least 90% towards retinol, are obtainable from animals including humans, preferably obtained from rat or human, such as human HsRDH12 (polypeptide sequence derived from NP_689656.2) or rat RnRDH12 (polypeptide sequence derived from NP_001101507.1), e.g. polypeptides with at least 60%, such as e.g. 65, 70, 75, 80, 85, 90, 95, 97, 98, 99% or up to 100% identity to a polypeptide encoded by SEQ ID NO:5 or 6.

In one embodiment, the host cell as described herein is capable of conversion of retinal with a total conversion of at least about 90%, preferably 92, 95, 97, 98, 99 or even 100% towards production of retinol. Preferably, such conversion is obtained from a retinal mix comprising a percentage of at least about 61% as trans-retinal, such as e.g. about 61 to 90% in trans-isoform, which is produced in the host cell. The retinal might be obtained via conversion of beta-carotene into retinal, catalyzed by respective beta-carotene oxidases (BCO), such as e.g. preferably the Drosophila melanogaster BCO, DmNinaB, or a polypeptide with at least 60%, such as e.g. 65, 70, 75, 80, 85, 90, 95, 97, 98, 99% or up to 100% identity to a polypeptide according to SEQ ID NO:3. Preferably, the retinal mix to be converted into retinol by the action of the RDH as defined herein comprises at least 61-65% trans-retinal, such as e.g. about 61 to 90% in trans-isoform, however, the activity/conversion of the RDHs according to the present invention is independent of the percentage of trans- and cis-retinal.

Thus, in one embodiment the invention is directed to a carotenoid-producing host cell, particularly fungal host cell, comprising (1) a stereoselective beta-carotene oxidase (BCO), i.e. a trans-specific BCO, catalyzing the conversion of beta-carotene into a mix of cis- and trans-retinal with a percentage of at least 61% trans-retinal in the retinal mix; and (2) a specific RDH as defined herein catalyzing the conversion of retinal, e.g. a retinal mix with a percentage of at least 61% trans-retinal based on the total amount of retinal in the mix, into retinol with a total conversion of at least about 90% towards retinol.

Examples of such BCOs as defined herein might be obtained from any source, such as e.g. plant, animal, bacteria, fungi, algae. Particular useful stereoselective BCOs are obtained from fungi, in particular Dikarya, including but not limited to fungi selected from Ascomycota or Basidiomycota, preferably obtained from Fusarium or Ustilago, more preferably isolated from F. fujikuroi or U. maydis, such as e.g. FfCarX (polypeptide sequence derived from AJ854252), UmCCO1 (polypeptide sequence derived from EAK81726). Furthermore, particularly useful stereoselective BCOs are obtained from insects, in particular Diptera, preferably obtained from Drosophila, more preferably from D. melanogaster, such as e.g. DmNinaB or DmBCO (polypeptide sequence derived from NP_650307.2). Furthermore, particularly useful stereoselective BCOs are obtained from plants, in particular Angiosperms, preferably obtained from Crocus, more preferably from C. sativus, such as e.g. CsZCO (polypeptide sequence derived from Q84K96.1). Furthermore, particularly useful stereoselective BCOs are obtained from eukaryotes, in particular pesces, preferably obtained from Danio or Ictalurus, more preferably from D. rerio or I. punctatus, such as e.g. DrBCO1 (polypeptide sequence derived from Q90WH4), IpBCO (polypeptide sequence derived from XP_017333634).

In one preferred aspect of the invention, a carotenoid-producing host cell, particularly fungal host cell, comprises (1) a stereoselective BCO which is selected from Drosophila, such as D. melanogaster, preferably a polypeptide according to SEQ ID NO:3, and (2) RDH having activity towards generation of retinol as defined herein which is selected from fungi, such as Fusarium, preferably from F. fujikuroi, more preferably FfRDH12 (SEQ ID NO:1).

Modifications in order to have the host cell as defined herein produce more copies of genes and/or proteins, such as e.g. trans-selective BCOs or RDHs with selectivity towards formation of retinol as defined herein, may include the use of strong promoters, suitable transcriptional- and/or translational enhancers, or the introduction of one or more gene copies into the carotenoid-producing host cell, leading to increased accumulation of the respective enzymes in a given time. The skilled person knows which techniques to use in dependence of the host cell. The increase or reduction of gene expression can be measured by various methods, such as e.g. Northern, Southern, or Western blot technology as known in the art.

The generation of a mutation into nucleic acids or amino acids, i.e. mutagenesis, may be performed in different ways, such as for instance by random or side-directed mutagenesis, physical damage caused by agents such as for instance radiation, chemical treatment, or insertion of a genetic element. The skilled person knows how to introduce mutations.

Thus, the present invention is directed to a carotenoid-producing host cell, particularly fungal host cell, as described herein comprising an expression vector or a polynucleotide encoding RDHs as described herein which has been integrated in the chromosomal DNA of the host. Such carotenoid-producing host cell, particularly fungal host cell, comprising a heterologous polynucleotide either on an expression vector or integrated into the chromosomal DNA encoding RDHs as described herein is called a recombinant host cell. The carotenoid-producing host cell, particularly fungal host cell, might contain one or more copies of a gene encoding the RDHs as defined herein, such as e.g. polynucleotides encoding polypeptides with at least about 60% identity to a polypeptide according to SEQ ID NO:1, leading to overexpression of such genes encoding the RDHs as defined herein. The increase of gene expression can be measured by various methods, such as e.g. Northern, Southern, or Western blot technology as known in the art.

Based on the sequences as disclosed herein and of the preference for reduction of retinal to retinol with a total conversion of at least about 90% one could easily deduce further suitable genes encoding polypeptides having retinal reducing activity as defined herein which could be used for the conversion of retinal into retinol, in particular wherein the percentage of trans-retinal in the retinal mix to be converted is at least about 61%, such as e.g. at least about 61 to 90% trans-retinal present in the retinal mix. Thus, the present invention is directed to a method for identification of novel retinal reducing enzymes, wherein a polypeptide with at least 60%, such as e.g. 65, 70, 75, 80, 85, 90, 95, 97, 98, 99% or up to 100% identity to F. fujikuroi RDH12 (SEQ ID NO:1) is used as a probed in a screening process for new retinal reducing enzymes with preference for production of retinol at total conversion of at least about 90%. Any polypeptide having RDH activity might be used for production of retinol from retinal as described herein, as long as the reductive action results in at least about 90% retinol compared to the amount retinal in the reaction mixture. Thus, a suitable RDH to be used for a process according to the present invention includes an enzyme capable to produce about 10% or less of retinal, such as e.g. based on the total amount of retinoids obtained from the conversion of retinol into retinal (backwards reaction).

The present invention is particularly directed to the use of such novel retinal reducing enzymes in a process for production of retinol, wherein the production of retinal by the action of said RDH as defined herein has been reduced or abolished and wherein the production of retinol has been increased, leading to a ratio between retinol and retinal in the retinoid mix of at least about 9:1. The process might be performed with a suitable carotenoid-producing host cell expressing said RDH, preferably wherein the genes encoding said RDHs are heterologous expressed, i.e. introduced into said host cells. Retinol, can be further converted into vitamin A by the action of (known) suitable mechanisms.

A reduced or abolished activity towards conversion of retinol into retinal as used herein means that the activity towards production of retinal is decreased relative to the enzymatic activity towards production of retinol. As used herein, reduction or abolishing the activity towards conversion of retinol into retinal, i.e. improvement of the product ratio towards reduction of retinal into retinol means a product ratio between retinol and retinal in the mix of retinoids which is at least about 9:1, such as e.g. 9.1:1, 9.2:1, 9.5:1, 9.8:1 or up to 10:1, which product ratios are achieved with the specific RDHs as defined herein.

A reduction or abolishment of the amount of retinal in the retinoid mix means a limitation to about 10% or less retinal in the retinoid mix based on the total amount of retinoids produced via enzymatic action of the RDH as defined herein.

The use of a retinal reducing enzyme as defined herein leads to an increase of at least about 18% in total conversion, such as e.g. at least about 20, 30, 40, 50, 60, 70, 80% compared to a non-modified host cell carrying (only) the endogenous RDHs, such in a fungal host cell, such as e.g. Yarrowia or Saccharomyces with no further genetical modification with regards to reduction of retinal, i.e. expressing the endogenous fungal RDH homologs present in the host cell.

As used herein, the term “at least about 90%” with regards to production of retinol, in particular with regards to production of retinol from conversion of retinal using a RDH as defined herein, means that at least about 90%, such as e.g. 92, 95, 98% or up to 100% of the retinal is converted into retinol. The term “about 10% or less” with regards to production of retinal, in particular with regards to production of retinal from conversion of retinol using an RDH defined herein, means that about 10% or less, such as e.g. 8, 7, 5, 2 or up to 0% of the produced retinol is converted back into retinal. All these numbers are based on the amount of retinal and retinol in the retinoid mixture present in a suitable carotenoid-producing host cell as defined herein.

The terms “sequence identity”, “% identity” or “sequence homology” are used interchangeable herein. For the purpose of this invention, it is defined here that in order to determine the percentage of sequence homology or sequence identity of two amino acid sequences or of two nucleic acid sequences, the sequences are aligned for optimal comparison purposes. In order to optimize the alignment between the two sequences gaps may be introduced in any of the two sequences that are compared. Such alignment can be carried out over the full length of the sequences being compared. Alternatively, the alignment may be carried out over a shorter length, for example over about 20, about 50, about 100 or more nucleic acids/bases or amino acids. The sequence identity is the percentage of identical matches between the two sequences over the reported aligned region. The percent sequence identity between two amino acid sequences or between two nucleotide sequences may be determined using the Needleman and Wunsch algorithm for the alignment of two sequences (Needleman, S. B. and Wunsch, C. D. (1970) J. Mol. Biol. 48, 443-453). Both amino acid sequences and nucleotide sequences can be aligned by the algorithm. The Needleman-Wunsch algorithm has been implemented in the computer program NEEDLE. For the purpose of this invention the NEEDLE program from the EMBOSS package was used (version 2.8.0 or higher, EMBOSS: The European Molecular Biology Open Software Suite (2000) Rice, Longden and Bleasby, Trends in Genetics 16, (6) pp 276-277, http://emboss.bioinformatics.nl/). For protein sequences EBLOSUM62 is used for the substitution matrix. For nucleotide sequence, EDNAFULL is used. The optional parameters used are a gap-open penalty of 10 and a gap extension penalty of 0.5. The skilled person will appreciate that all these different parameters will yield slightly different results but that the overall percentage identity of two sequences is not significantly altered when using different algorithms.

After alignment by the program NEEDLE as described above the percentage of sequence identity between a query sequence and a sequence of the invention is calculated as follows: number of corresponding positions in the alignment showing an identical amino acid or identical nucleotide in both sequences divided by the total length of the alignment after subtraction of the total number of gaps in the alignment. The identity as defined herein can be obtained from NEEDLE by using the NOBRIEF option and is labeled in the output of the program as “longest identity”. If both amino acid sequences which are compared do not differ in any of their amino acids, they are identical or have 100% identity. With regards to enzymes originated from plants as defined herein, the skilled person knows plant-derived enzymes might contain a chloroplast targeting signal which is to be cleaved via specific enzymes, such as e.g. chloroplast processing enzymes (CPEs).

Depending on the host cell, the polynucleotides as defined herein might be optimized for expression in the respective host cell. The skilled person knows how to generate such modified polynucleotides. It is understood that the polynucleotides as defined herein also encompass such host-optimized nucleic acid molecules as long as they still express the polypeptide with the respective activities as defined herein.

Thus, in one embodiment, the present invention is directed to a carotenoid-producing host cell, particularly fungal host cell, comprising polynucleotides encoding RDHs as defined herein which are optimized for expression in said host cell, with no impact on growth of expression pattern of the host cell or the enzymes. Particularly, a carotenoid-producing host cell, particularly fungal host cell, is selected from Yarrowia, such as Yarrowia lipolytica, wherein the polynucleotides encoding the RDHs as defined herein are selected from polynucleotides with at least 60%, such as e.g. 65, 70, 75, 80, 85, 90, 92, 95, 97, 98, 99% or up to 100% identity to SEQ ID NOs:2, 5, 6, or 7.

The RDHs as defined herein also encompasses enzymes carrying amino acid substitution(s) which do not alter enzyme activity, i.e. which show the same properties with respect to the wild-type enzyme and catalyze the conversion of retinal to retinol, in particular with a total conversion of at least about 90% towards production of retinol. Such mutations are also called “silent mutations”, which do not alter the (enzymatic) activity of the enzymes as described herein.

A nucleic acid molecule according to the invention may comprise only a portion or a fragment of the nucleic acid sequence provided by the present invention, such as for instance polynucleotide sequences or according to SEQ ID NOs: 2, 4, 5, 6 or 7 for example a fragment which may be used as a probe or primer or a fragment encoding a portion of a RDH as defined herein. The nucleotide sequence determined from the cloning of the RDH gene allows for the generation of probes and primers designed for use in identifying and/or cloning other homologues from other species. The probe/primer typically comprises substantially purified oligonucleotides which typically comprises a region of nucleotide sequence that hybridizes preferably under highly stringent conditions to at least about 12 or 15, preferably about 18 or 20, more preferably about 22 or 25, even more preferably about 30, 35, 40, 45, 50, 55, 60, 65, or 75 or more consecutive nucleotides of a nucleotide sequence according to SEQ ID NOs: 2, 4, 5, 6 or 7 or a fragment or derivative thereof.

A preferred, non-limiting example of such hybridization conditions are hybridization in 6× sodium chloride/sodium citrate (SSC) at about 45° C., followed by one or more washes in 1×SSC, 0.1% SDS at 50° C., preferably at 55° ° C., more preferably at 60° C. and even more preferably at 65° C.

Highly stringent conditions include, for example, 2 h to 4 days incubation at 42° C. using a digoxigenin (DIG)-labeled DNA probe (prepared by using a DIG labeling system; Roche Diagnostics GmbH, 68298 Mannheim, Germany) in a solution such as DigEasyHyb solution (Roche Diagnostics GmbH) with or without 100 μg/ml salmon sperm DNA, or a solution comprising 50% formamide, 5×SSC (150 mM NaCl, 15 mM trisodium citrate), 0.02% sodium dodecyl sulfate, 0.1% N-lauroylsarcosine, and 2% blocking reagent (Roche Diagnostics GmbH), followed by washing the filters twice for 5 to 15 minutes in 2×SSC and 0.1% SDS at room temperature and then washing twice for 15-30 minutes in 0.5×SSC and 0.1% SDS or 0.1×SSC and 0.1% SDS at 65-68° C.

Expression of the enzymes/polynucleotides encoding one of the specific RDHs as defined herein can be achieved in any host system, including (micro)organisms, which is suitable for carotenoid/retinoid production and which allows expression of the nucleic acids encoding one of the enzymes as disclosed herein, including functional equivalents or derivatives as described herein. Examples of suitable carotenoid/retinoid-producing host (micro)organisms are bacteria, algae, fungi, including yeasts, plant or animal cells. Preferred bacteria are those of the genera Escherichia, such as, for example, Escherichia coli, Streptomyces, Pantoca (Erwinia), Bacillus, Flavobacterium, Synechococcus, Lactobacillus, Corynebacterium, Micrococcus, Mixococcus, Brevibacterium, Bradyrhizobium, Gordonia, Dietzia, Muricauda, Sphingomonas, Synochocystis, Paracoccus, such as, for example, Paracoccus zeaxanthinifaciens. Preferred eukaryotic microorganisms, in particular fungi including yeast, are selected from Saccharomyces, such as Saccharomyces cerevisiae, Aspergillus, such as Aspergillus niger, Pichia, such as Pichia pastoris, Hansenula, such as Hansenula polymorpha, Phycomyces, such as Phycomyces blakesleanus, Mucor, Rhodotorula, Sporobolomyces, Xanthophyllomyces, Phaffia, Blakeslea, such as e.g. Blakeslea trispora, or Yarrowia, such as Yarrowia lipolytica. In particularly preferred is expression in a fungal host cell, such as e.g. Yarrowia or Saccharomyces, or expression in Escherichia, more preferably expression in Yarrowia lipolytica or Saccharomyces cerevisiae.

With regards to the present invention, it is understood that organisms, such as e.g. microorganisms, fungi, algae, or plants also include synonyms or basonyms of such species having the same physiological properties, as defined by the International Code of Nomenclature of Prokaryotes or the International Code of Nomenclature for algae, fungi, and plants (Melbourne Code).

As used herein, a carotenoid-producing host cell, particularly fungal host cell, is a host cell, wherein the respective polypeptides are expressed and active in vivo leading to production of carotenoids, e.g. beta-carotene. The genes and methods to generate carotenoid-producing host cells are known in the art, see e.g. WO2006102342. Depending on the carotenoid to be produced, different genes might be involved.

As used herein, a retinoid-producing host cell, particularly fungal host cell, is a host cell wherein, the respective polypeptides are expressed and active in vivo, leading to production of retinoids, e.g. vitamin A and its precursors, via enzymatic conversion of beta-carotene via retinal and retinol. These polypeptides include the RDHs as defined herein. The genes of the vitamin A pathway and methods to generate retinoid-producing host cells are known in the art. Preferably, the beta-carotene is converted into retinal via action of beta-carotene oxidizing enzymes, the retinal is further converted into retinol via action of RDHs as defined herein, and the retinol is converted into retinol acetate via action of acetyl-transferase enzymes, such as e.g. ATF1. The retinol acetate might be the retinoid of choice which is isolated from the host cell.

The present invention is directed to a process for production of retinol, in particular with a total conversion of at least 90%, via reduction of retinal by the action of a RDH as described herein, wherein the amount of retinal in the produced retinoid mix is about 10% or less, wherein the retinal reducing enzymes are preferably heterologous expressed in a suitable host cell under suitable conditions as described herein. The produced retinol might be isolated and optionally further purified from the medium and/or host cell. In a further embodiment, retinol can be used as precursor in a multi-step process leading to vitamin A. Vitamin A might be isolated and optionally further purified from the medium and/or host cell as known in the art.

Thus, the present invention is directed to a process for decreasing the percentage of retinal in a retinoid mix, or for increasing the percentage of retinol in a retinoid mix, wherein the retinol is generated via contacting one of the RDHs as defined herein with retinal, resulting in a retinol/retinal-mix with a percentage of at least about 90% retinol or about 35% or less of retinal. Particularly, said process comprising (a) introducing a nucleic acid molecule encoding one of the RDHs as defined herein into a suitable carotenoid-producing host cell, particularly fungal host cell, as defined herein, (b) enzymatic cleavage of retinal into retinol via action of said expressed RDH wherein the percentage of retinol is at least 90% based on the total amount of retinal and retinol in the retinoid mix, and optionally (3) conversion of retinol, preferably trans-retinol, into vitamin A under suitable conditions known to the skilled person.

The host cell, i.e. microorganism, algae, fungal, animal or plant cell, which is able to express the beta-carotene producing genes, the RDH genes as defined herein, optionally the genes encoding beta-carotene oxygenating enzymes and/or optionally further genes required for biosynthesis of vitamin A, may be cultured in an aqueous medium supplemented with appropriate nutrients under aerobic or anaerobic conditions and as known by the skilled person for the different host cells. Optionally, such cultivation is in the presence of proteins and/or co-factors involved in transfer of electrons, as defined herein. The cultivation/growth of the host cell may be conducted in batch, fed-batch, semi-continuous or continuous mode. Depending on the host cell, preferably, production of retinoids such as e.g. vitamin A and precursors such as retinal, retinol can vary, as it is known to the skilled person. Cultivation and isolation of beta-carotene and retinoid-producing host cells selected from Yarrowia is described in e.g. WO2008042338. With regards to production of retinoids in host cells selected from E. coli, methods are described in e.g. Jang et al, Microbial Cell Factories, 10:95 (2011). Specific methods for production of beta-carotene and retinoids in yeast host cells, such as e.g. Saccharomyces cerevisiae, are disclosed in e.g. WO2014096992.

As used herein, the term “specific activity” or “activity” with regards to enzymes means its catalytic activity, i.e. its ability to catalyze formation of a product from a given substrate. The specific activity defines the amount of substrate consumed and/or product produced in a given time period and per defined amount of protein at a defined temperature. Typically, specific activity is expressed in μmol substrate consumed or product formed per min per mg of protein. Typically, μmol/min is abbreviated by U (=unit). Therefore, the unit definitions for specific activity of μmol/min/(mg of protein) or U/(mg of protein) are used interchangeably throughout this document. An enzyme is active, if it performs its catalytic activity in vivo, i.e. within the host cell as defined herein or within a system in the presence of a suitable substrate. The skilled person knows how to measure enzyme activity, in particular activity of RDHs as defined herein. Analytical methods to evaluate the capability of a suitable RDH as defined herein for retinol production from conversion of retinal are known in the art, such as e.g. described in Example 4 of WO2014096992. In brief, titers of products such as retinol, trans-retinal, cis-retinal, beta-carotene and the like can be measured by HPLC.

Retinoids as used herein include beta-carotene cleavage products also known as apocarotenoids, including but not limited to retinal, retinolic acid, retinol, retinoic methoxide, retinyl acetate, retinyl esters, 4-keto-retinoids, 3 hydroxy-retinoids or combinations thereof. A mixture comprising retinal and retinol is referred to herein as “retinoid mix”, wherein the percentage “at least about 90%” with regards to retinol or “about 10% or less” with regards to retinal refers to the ratio of retinol to retinal in such retinoid mix. Biosynthesis of retinoids is described in e.g. WO2008042338.

Retinal as used herein is known under IUPAC name (2E,4E,6E,8E)-3,7-Dimethyl-9-(2,6,6-trimethylcyclohexen-1-yl)nona-2,4,6,8-tetraenal. It is herein interchangeably referred to as retinaldehyde or vitamin A aldehyde and includes both cis- and trans-isoforms, such as e.g. 11-cis retinal, 13-cis retinal, trans-retinal and all-trans retinal.

The term “carotenoids” as used herein is well known in the art. It includes long, 40 carbon conjugated isoprenoid polyenes that are formed in nature by the ligation of two 20 carbon geranylgeranyl pyrophosphate molecules. These include but are not limited to phytoene, lycopene, and carotene, such as e.g. beta-carotene, which can be oxidized on the 4-keto position or 3-hydroxy position to yield canthaxanthin, zeaxanthin, or astaxanthin. Biosynthesis of carotenoids is described in e.g. WO2006102342.

Vitamin A as used herein may be any chemical form of vitamin A found in aqueous solutions, such as for instance undissociated, in its free acid form or dissociated as an anion. The term as used herein includes all precursors or intermediates in the biotechnological vitamin A pathway. It also includes vitamin A acetate.

In particular, the present invention features the present embodiments:
A carotenoid-producing host cell, particularly fungal host cell, comprising a retinol dehydrogenase [EC 1.1.1.105], said host cell producing a retinoid mix comprising retinal and retinol, wherein the percentage of retinol is at least about 90%, preferably 92, 95, 97, 98, 99 or even 100% compared to the amount of retinal present in said retinoid mix.
The carotenoid-producing host cell, particularly fungal host cell, as above and defined herein, wherein the retinal to be reduced via action of the retinol dehydrogenase comprises a mix of trans-retinal and cis-retinal, wherein the percentage of trans-retinal in said retinal mix is in the range of at least about 61 to 98%, preferably at least about 61 to 95%, more preferably at least about 61 to 90%.
The carotenoid-producing host cell, particularly fungal host cell, as above and defined herein, comprising a heterologous retinol dehydrogenase.
The carotenoid-producing host cell as above and defined herein, wherein the host cell is selected from plants, fungi, algae or microorganisms, preferably selected from fungi including yeast, more preferably from Saccharomyces, Aspergillus, Pichia, Hansenula, Phycomyces, Mucor, Rhodotorula, Sporobolomyces, Xanthophyllomyces, Phaffia, Blakeslea or Yarrowia, even more preferably from Yarrowia lipolytica or Saccharomyces cerevisiae.
The carotenoid-producing host cell as above and defined herein, wherein the host cell is selected from plants, fungi, algae or microorganisms, preferably selected from Escherichia, Streptomyces, Pantoca, Bacillus, Flavobacterium, Synechococcus, Lactobacillus, Corynebacterium, Micrococcus, Mixococcus, Brevibacterium, Bradyrhizobium, Gordonia, Dietzia, Muricauda, Sphingomonas, Synochocystis or Paracoccus.
The carotenoid-producing host cell, particularly fungal host cell, as above and defined herein, wherein the retinol dehydrogenase is selected from fungi, preferably Fusarium, more preferably Fusarium fujikuroi, most preferably selected from the group F. fujikuroi RDH12, particularly selected from a polypeptide with at least 60% identity to a polypeptide according to SEQ ID NO: 1 or sequences encoded by a polynucleotide according to SEQ ID NO:2.
The carotenoid-producing host cell as above and defined herein, wherein the retinol is further converted into vitamin A.
The carotenoid-producing host cell, particularly fungal host cell, as above and defined herein, further comprising a trans-selective beta-carotene oxidizing enzyme selected from Drosophila catalyzing the conversion of beta-carotene to a retinal mix, wherein the mix comprises at least about 61%, preferably 65, 68, 70, 75, 80, 85, 90, 95, 98 or up to 100% retinal in trans-isoform based on the total amount of retinal in the mix, more preferably selected from a sequence with at least 60% identity to a polypeptide according to SEQ ID NO:3.
A process for production of a retinoid mix comprising retinol and retinal via enzymatic activity of a retinol dehydrogenase [EC 1.1.1.105], comprising contacting retinal with said retinol dehydrogenase, wherein the ratio of retinol to retinal in the retinoid mix is at least about 9:1.
A process for decreasing the amount of retinal in a retinoid mix produced from enzymatic action of retinol dehydrogenase, said process comprising contacting retinal with a retinol dehydrogenase as defined herein, wherein the amount of retinal in the retinoid mix resulting from said enzymatic action is in the range of about 10% or less compared to the amount of retinol.
A process for increasing the amount of retinol in a retinoid mix produced from enzymatic action of retinol dehydrogenase, said process comprising contacting retinal with a retinol dehydrogenase as defined herein, wherein the amount of retinol in the retinoid mix resulting from said enzymatic action is in the range of at least about 90% compared to the amount of retinol.
A process according as above and defined herein using the carotenoid-producing host cell, particularly fungal host cell, comprising a retinol dehydrogenase [EC 1.1.1.105], said host cell producing a retinoid mix comprising retinal and retinol, wherein the percentage of retinol is at least about 90%, preferably 92, 95, 97, 98, 99 or even 100% compared to the amount of retinal present in said retinoid mix.
A process for production of vitamin A comprising the steps of:
(a) introducing a nucleic acid molecule encoding a retinol dehydrogenase [EC 1.1.1.105] as defined herein into a suitable carotene-producing host cell, particularly fungal host cell,
(b) enzymatic conversion of retinal into a retinoid mix comprising retinol and retinal in a ratio of at least about 9:1,
(c) conversion of retinol into vitamin A under suitable culture conditions.
Use of a retinol dehydrogenase [EC 1.1.1.105] as above and defined herein for production of a retinoid mix comprising retinol and retinal in a ratio of 9:1, wherein the retinol dehydrogenase is heterologous expressed in a suitable carotenoid-producing host cell, particularly fungal host cell.

The following examples are illustrative only and are not intended to limit the scope of the invention in any way. The contents of all references, patent applications, patents, and published patent applications, cited throughout this application are hereby incorporated by reference, in particular WO2006102342, WO2008042338 or WO2014096992.

### EXAMPLES

### Example 1: General Methods, Strains, and Plasmids

All basic molecular biology and DNA manipulation procedures described herein are generally performed according to Sambrook et al. (eds.), Molecular Cloning: A Laboratory Manual. Cold Spring Harbor Laboratory Press: New York (1989) or Ausubel et al. (eds). Current Protocols in Molecular Biology. Wiley: New York (1998).

Shake plate assay. Typically, 800 μl of 0.075% Yeast extract, 0.25% peptone (0.25× YP) is inoculated with 10 μl of freshly grown Yarrowia and overlaid with 200 μl of Drakcol 5 mineral oil carbon source 5% corn oil in mineral oil and/or 5% in glucose in aqueous phase. Transformants were grown in 24 well plates (Multitron, 30° ° C., 800 RPM) in YPD media with 20% dodecane for 4 days. The mineral oil fraction was removed from the shake plate wells and analyzed by HPLC on a normal phase column, with a photo-diode array detector.

DNA transformation. Strains are transformed by overnight growth on YPD plate media 50 μl of cells is scraped from a plate and transformed by incubation in 500 μl with 1 μg transforming DNA, typically linear DNA for integrative transformation, 40% PEG 3550MW, 100 mM lithium acetate, 50 mM Dithiothreitol, 5 mM Tris-Cl pH 8.0, 0.5 mM EDTA for 60 minutes at 40° C. and plated directly to selective media or in the case of dominant antibiotic marker selection the cells are out grown on YPD liquid media for 4 hours at 30° ° C. before plating on the selective media.

DNA molecular biology. Genes were synthesized with NheI and MluI ends in pUC57 vector. Typically, the genes were subcloned to the MB5082 ‘URA3’, MB6157 HygR, and MB8327 NatR vectors for marker selection in Yarrowia lipolytica transformations, as in WO2016172282. For clean gene insertion by random nonhomologous end joining of the gene and marker HindIII/XbaI (MB5082) or PvuII (MB6157 and MB8327), respectively purified by gel electrophoresis and Qiagen gel purification column.

Plasmid list. Plasmid, strains, nucleotide and amino acid sequences to be used are listed are listed in Table 1, 2 and the sequence listing. Nucleotide sequence ID NOs:2, 4, 5, 6, and 7 are codon optimized for expression in Yarrowia.

TABLE 1
list of plasmids used for construction of the strains carrying the
heterologous RDH-genes. The sequence ID NOs refer to the inserts.
For more details, see text.

| | | | SEQ ID NO: |
|---|---|---|---|
| MB plasmid | Backbone MB | Insert | (aa/nt) |
| 8200 | 5082 | FfRDH12 | 1/2 |
| 8203 | 5082 | HsRDH12 | 5 |
| 8196 | 5082 | RnRDH12 | 6 |
| 8197 | 5082 | McRDH12 | 7 |

TABLE 2
list of Yarrowia strains used for production of retinoids carrying the
heterologous RDH genes. For more details, see text.

| ML strain | Description | First described in |
|---|---|---|
| 7788 | Carotene strain | WO2016172282 |
| 15710 | ML7788 transformed with | WO2016172282 |
| | MB7311-Mucor CarG | |
| 17544 | ML15710 cured of URA3 by | here |
| | FOA and HygR by Cre/lox | |
| 17767 | ML17544 transformed with | here |
| | MB6072 DmBCO-URA3 and | |
| | MB6732 SbATF1-HygR and | |
| | cured of markers | |
| 17978 | ML17968 transformed with | here |
| | MB8200 FfRDH-URA3 and | |
| | cured of markers | |

Normal phase retinol method. A Waters 1525 binary pump attached to a Waters 717 auto sampler were used to inject samples. A Phenomenex Luna 3μ Silica (2), 150×4.6 mm with a security silica guard column kit was used to resolve retinoids. The mobile phase consists of either, 1000 mL hexane, 30 mL isopropanol, and 0.1 mL acetic acid for astaxanthin related compounds, or 1000 mL hexane, 60 mL isopropanol, and 0.1 mL acetic acid for zeaxanthin related compounds. The flow rate for each is 0.6 mL per minute. Column temperature is ambient. The injection volume is 20 μL. The detector is a photodiode array detector collecting from 210 to 600 nm. Analytes were detected according to Table 3.

TABLE 3
list of analytes using normal phase retinol method.
For more details, see text.

| | Retention | Lambda |
|---|---|---|
| | time | max |
| Intermediates | [min] | [nm] |
| 11-cis-dihydro-retinol | 7.1 | 293 |
| 11-cis-retinal | 4 | 364 |
| 11-cis-retinol | 8.6 | 318 |
| 13-cis-retinal | 4.1 | 364 |
| dihydro-retinol | 9.2 | 292 |
| retinyl-acetate | 3.5 | 326 |
| retinyl-ester | 3 | 325 |
| trans-retinal | 4.7 | 376 |
| trans-retinol | 10.5 | 325 |

Sample preparation. Samples were prepared by various methods depending on the conditions. For whole broth or washed broth samples the broth was placed in a Precellys® tube weighed and mobile phase was added, the samples were processed in a Precellys® homogenizer (Bertin Corp, Rockville, MD, USA) on the highest setting 3× according to the manufactures directions. In the washed broth the samples were spun in a 1.7 ml tube in a microfuge at 10000 rpm for 1 minute, the broth decanted, 1 ml water added mixed pelleted and decanted and brought up to the original volume the mixture was pelleted again and brought up in appropriate amount of mobile phase and processed by Precellys® bead beating. For analysis of mineral oil fraction, the sample was spun at 4000 RPM for 10 minutes and the oil was decanted off the top by positive displacement pipet (Eppendorf, Hauppauge, NY, USA) and diluted into mobile phase mixed by vortexing and measured for retinoid concentration by HPLC analysis.

Fermentation conditions. Fermentations were identical to the previously described conditions using mineral oil overlay and stirred tank that was corn oil fed in a bench top reactor with 0.5 L to 5 L total volume (see WO2016172282). Generally, the same results were observed with a fed batch stirred tank reactor with an increased productivity demonstrating the utility of the system for the production of retinoids.

### Example 2: Production of Retinoids in Yarrowia lipolytica

Typically, the beta carotene strain ML17767 was transformed with purified HinDIII/XbaI fragments derived from plasmids containing retinol dehydrogenase (RDH) gene fragments linker to a URA3 promoter. Six to eight isolates were screened for a decrease in the retinol:retinal ratio in a shake plate assay and successful isolates were run in a fed batch stirred tank reactor for eight days which showed an order of magnitude increase in the productivity of the process which indicates a utility in large scale production. The best results were obtained with the Fusarium RDH12 homolog with only 2% or residual retinal maintained after 8 days of shake-flask incubation as described above. The isolate derived from the Fusarium sequence demonstrated an increased reduction of retinol as indicated in the following table.

### Example 3: Production of Retinoids in Saccharomyces cerevisiae

Typically, a beta carotene strain is transformed with heterologous genes encoding for enzymes such as geranylgeranyl synthase, phytoene synthase, lycopene synthase, lycopene cyclase constructed that is producing beta carotene according to standard methods as known in the art (such as e.g. as described in US20160130628 or WO2009126890). Further, when transformed with beta carotene oxidase genes retinal can be produced. Further, when transformed with retinol dehydrogenase, then retinol can be produced. With this approach, similar results regarding specificity for productivity towards retinol are obtained.

### Example 4: Production of Retinol from Beta-Carotene

In addition to the single modifications described in Examples 2, 3 and 4 a strain was constructed carrying the heterologous together with the heterologous FtRDH12. Fermentation and analysis of the retinoids was done as described before.

For expression of heterologous BCO from Drosophila melanogaster DmNinaB (DmBCO1; SEQ ID NO:3), a beta carotene strain ML17544 was transformed with purified linear DNA fragment by HindII and XbaI mediated restriction endonucleotide cleavage of beta carotene oxidase (BCO) containing codon optimized fragments linked to a URA3 nutritional marker. Transforming DNA were derived from MB6702 Drosophila NinaB BCO gene, whereby the codon-optimized sequences (SEQ ID NO:4) had been used. The gene were then grown screening 6-8 isolates in a shake plate analysis, isolates that performed well were run in a fed batch stirred tank reaction for 8-10 days. Detection of cis- and trans-retinal was made by HPLC using standard parameters as described in WO2014096992, but calibrated with purified standards for the retinoid analytes. The amount of trans-retinal in the retinal mix heterologous expressing the BCO from Drosophila melanogaster (SEQ ID NO:3) resulted in 61% of trans-retinal based on the total amount of retinal (not shown).

The presence of heterologous FtRDH12 reduced the amount of retinal detected in the analyte from 20% to 4%, which is a good indication for specific retinal-reducing activity of the Fusarium RDH12 (see Ex. 2), with still a percentage of trans-retinol in the range of at least 61%.

1. A carotenoid-producing host cell comprising a retinol dehydrogenase [EC 1.1.1.105], preferably a heterologous retinol dehydrogenase, said host cell producing a retinoid mix comprising retinal and retinol, wherein the percentage of retinol is at least about 90%, preferably 92, 95, 97, 98, 99 or even 100% compared to the amount of retinal present in said retinoid mix.

2. The carotenoid-producing host cell of claim 1, wherein the retinal to be reduced via action of the retinol dehydrogenase comprises a mix of trans-retinal and cis-retinal, wherein the percentage of trans-retinal in said retinal mix is in the range of at least about 61 to 98%, preferably at least about 61 to 95%, more preferably at least about 61 to 90%.

3. The carotenoid-producing host cell according to claim 1, wherein the retinol dehydrogenase is selected from fungi, preferably Fusarium, more preferably retinol dehydrogenase is a Fusarium fujikuroi retinol dehydrogenase (FtRDH).

4. The carotenoid-producing host cell according to claim 3, wherein the FtRDH is selected from a polypeptide with at least about 60% identity to a polypeptide according to SEQ ID NO:1.

5. The carotenoid-producing host cell according to claim 1, wherein the host cell is selected from plants, fungi, algae or microorganisms, such as selected from the group consisting of Escherichia, Streptomyces, Pantoea, Bacillus, Flavobacterium, Synechococcus, Lactobacillus, Corynebacterium, Micrococcus, Mixococcus, Brevibacterium, Bradyrhizobium, Gordonia, Dietzia, Muricauda, Sphingomonas, Synochocystis, Paracoccus, Saccharomyces, Aspergillus, Pichia, Hansenula, Phycomyces, Mucor, Rhodotorula, Sporobolomyces, Xanthophyllomyces, Phaffia, and Blakeslea, preferably selected from fungi including yeast, more preferably selected from the group consisting of Saccharomyces, Aspergillus, Pichia, Hansenula, Phycomyces, Mucor, Rhodotorula, Sporobolomyces, Xanthophyllomyces, Phaffia, Blakeslea and Yarrowia, most preferably from Yarrowia lipolytica or Saccharomyces cerevisiae.

6. The carotenoid-producing host cell according to claim 1, wherein the retinol is further converted into vitamin A.

7. The carotenoid-producing host cell according to claim 1, further comprising a stereoselective beta-carotene oxidizing enzyme selected from Drosophila catalyzing the conversion of beta-carotene to a retinal mix, wherein the mix comprises at least about 61%, preferably 68, 70, 75, 80, 85, 90, 95, 98 or up to 100% retinal in trans-isoform based on the total amount of retinal in the mix, more preferably selected from a sequence with at least 60% identity to a polypeptide according to SEQ ID NO:3.

8. A process for production of a retinoid mix comprising retinol and retinal via enzymatic activity of a retinol dehydrogenase [EC 1.1.1.105], comprising contacting retinal with said retinol dehydrogenase, wherein the ratio of retinol to retinal in the retinoid mix is at least about 9:1.

9. (canceled)

10. (canceled)

11. A process using the carotenoid-producing host cell according to claim 1.

12. A process for production of vitamin A comprising the steps of:
(a) introducing a nucleic acid molecule encoding a retinol dehydrogenase [EC 1.1.1.105] into a suitable carotene-producing host cell,
(b) enzymatic conversion of retinal into a retinoid mix comprising retinol and retinal in a ratio of at least about 9:1,
(c) conversion of retinol into vitamin A under suitable culture conditions.

13. Use of a carotenoid-producing host cell according to claim 1 for production of a retinoid mix comprising retinol and retinal in a ratio of 9:1.


SEQUENCE LISTING (7 sequences)

SEQ ID NO: 1 - protein, 325 aa, unidentified
MTTKYTSVHESPNGPGDARPTASQIIDDYNLEGELSGKTVLVTGCSSGIGVETARAIYRTGATLYLTARDVDKAKTVLPDLVDTSRVHFLHLDLNSLESVRGFAENFKSKSTQLHILIENAGVMACPEGRTVDGFETQFGINHLAHFLLFYLLKDTLLNSSTPAFNSRVVILSSCAHQAGSVHLNNLSLEGGYEPWKSYGQSKTANLWTAREIEKRFGASGIHSWAVHPGSIATELQRHVSDELKQKWADDKEGAKLWKSTEQGAATTVLAAVSPELEGKGGLYLEDTQVAKPPARGMFGVADWAYDEDGPSKLWAKSLELLKLQ

SEQ ID NO: 2 - DNA, 978 bp, synthetic construct
  misc_feature 1..978: note = Yarrowia codon-optimized synthetic polynucleotide
  source 1..978: mol_type = other DNA
atgaccactaagtacacttccgttcacgagtctcccaacggccctggtgacgctcgacccaccgcttcccagattatcgacgattacaaccttgagggagagctttctggcaagactgttctcgtcaccggctgttcctctggtattggtgttgagactgcccgagctatttaccgaactggtgccaccctttacctcactgcccgagatgtcgataaggccaagaccgttcttcccgaccttgttgacacttcccgagtccactttctccaccttgaccttaactctctggagtctgttcgaggttttgctgagaacttcaagtctaagtccactcagcttcacattctcatcgagaacgctggcgtgatggcctgtcccgagggccgaaccgtcgatggttttgagactcagtttggtatcaaccaccttgctcactttctcctcttttacctcctcaaggatacccttctcaactcttctacccccgctttcaactcccgagttgtcatcctctcttcttgtgctcaccaggctggttccgttcaccttaacaacctgtctcttgagggtggatacgagccttggaagtcttacggccagtccaagactgccaacctttggactgcccgagagatcgagaagcgatttggtgcttccggtatccactcttgggctgttcaccccggttccatcgctactgagcttcagcgacacgtttccgacgagcttaagcagaagtgggctgacgataaggagggtgccaagctgtggaagtccaccgagcagggtgccgccaccactgtccttgctgctgtttcccctgagcttgagggtaagggcggtctttaccttgaggatacccaggttgccaagccccctgcccgaggaatgtttggtgttgctgactgggcttacgatgaggatggcccctctaagctctgggccaagtctcttgagctccttaagctccagtaa

SEQ ID NO: 3 - protein, 620 aa, Drosophila melanogaster
MAAGVFKSFMRDFFAVKYDEQRNDPQAERLDGNGRLYPNCSSDVWLRSCEREIVDPIEGHHSGHIPKWICGSLLRNGPGSWKVGDMTFGHLFDCSALLHRFAIRNGRVTYQNRFVDTETLRKNRSAQRIVVTEFGTAAVPDPCHSIFDRFAAIFRPDSGTDNSMISIYPFGDQYYTFTETPFMHRINPCTLATEARICTTDFVGVVNHTSHPHVLPSGTVYNLGTTMTRSGPAYTILSFPHGEQMFEDAHVVATLPCRWKLHPGYMHTFGLTDHYFVIVEQPLSVSLTEYIKAQLGGQNLSACLKWFEDRPTLFHLIDRVSGKLVQTYESEAFFYLHIINCFERDGHVVVDICSYRNPEMINCMYLEAIANMQTNPNYATLFRGRPLRFVLPLGTIPPASIAKRGLVKSFSLAGLSAPQVSRTMKHSVSQYADITYMPTNGKQATAGEESPKRDAKRGRYEEENLVNLVTMEGSQAEAFQGTNGIQLRPEMLCDWGCETPRIYYERYMGKNYRYFYAISSDVDAVNPGTLIKVDVWNKSCLTWCEENVYPSEPIFVPSPDPKSEDDGVILASMVLGGLNDRYVGLIVLCAKTMTELGRCDFHTNGPVPKCLHGWFAPNAI

SEQ ID NO: 4 - DNA, 1863 bp, synthetic construct
  misc_feature 1..1863: note = Yarrowia codon-optimized synthetic polynucleotide
  source 1..1863: mol_type = other DNA
atggccgctggtgttttcaagtcttttatgcgagatttctttgctgttaagtacgatgagcagcgaaacgacccccaggccgagcgactggacggcaacggacgactgtaccccaactgctcctctgatgtttggcttcgatcttgcgagcgagagatcgttgaccccattgagggccaccactccggtcacattcccaagtggatttgcggttccctgctccgaaacggccccggctcttggaaggttggcgacatgaccttcggccacctgttcgactgctccgccctgctccaccgatttgccattcgaaacggacgagtcacctaccagaaccgatttgttgacactgagactctgcgaaagaaccgatctgcccagcgaattgttgtcaccgagtttggcactgccgctgttcccgatccctgtcactccatcttcgaccgatttgccgccatttttcgacccgattctggaaccgataactccatgatttccatctaccccttcggcgaccagtactacactttcaccgagactccctttatgcaccgaattaacccctgcactctcgctactgaggctcgaatctgcaccaccgacttcgttggcgttgtcaaccacacttctcacccccacgttcttccctctggcactgtttacaacctgggcaccactatgacccgatctggacccgcttacactatcctctctttcccccacggcgagcagatgttcgaggacgctcacgttgtcgccactctgccctgccgatggaagctgcaccccggttatatgcacaccttcggcctcactgaccactactttgtcattgttgagcagcccctttccgtttccctcactgagtacatcaaggcccagcttggcggacagaacctttccgcttgcctcaagtggttcgaggaccgacccactctctttcaccttattgatcgagtttccggcaagctggtccagacctacgagtccgaggctttcttctacctgcacatcatcaactgctttgagcgagatggccacgttgtcgttgacatttgctcttaccgaaaccccgagatgattaactgcatgtacctggaggccattgccaacatgcagactaaccccaactacgctaccctctttcgaggacgaccccttcgattcgtcctgcccctcggcactattccccccgcctctatcgccaagcgaggactcgtcaagtccttctccctcgctggactctccgctccccaggtttctcgaaccatgaagcactccgtttctcagtacgccgatattacctacatgcccaccaacggaaagcaggccactgctggagaggagtcccccaagcgagatgccaagcgaggccgatacgaggaggagaaccttgtcaacctggttactatggagggctctcaggccgaggcttttcagggcaccaacggcattcagcttcgacccgagatgctgtgtgattggggctgtgagactccccgaatctactacgagcgatacatgggcaagaactaccgatacttctacgccatttcttccgatgttgatgctgtcaaccccggcaccctcatcaaggttgatgtctggaacaagtcttgtcttacctggtgcgaggagaacgtctacccctctgagcccatttttgtcccctctcccgatcccaagtccgaggacgatggcgttatcctggcctctatggttcttggcggtcttaacgaccgatacgtcggccttattgttctttgtgccaagaccatgaccgagctgggccgatgtgatttccacaccaacggacccgttcccaagtgcctccacggttggtttgctcccaacgccatttag

SEQ ID NO: 5 - DNA, 900 bp, synthetic construct
  misc_feature 1..900: note = Yarrowia codon-optimized synthetic polynucleotide
  source 1..900: mol_type = other DNA
atggctccctccattcgaaagttctttgctggtggtgtgtgtcgaactaacgttcagcttcccggtaaggtggttgtcatcactggtgccaacaccggcattggcaaggagactgcccgagagctcgcttcccgaggagcccgagtttacattgcttgccgagatgttctgaagggcgagtctgctgcctctgagattcgagttgacactaagaactcccaggtgctcgtgcgaaagctcgacctttccgacactaagtctatccgagcctttgctgagggctttctcgctgaggagaagcagcttcacattctgattaacaacgctggagttatgatgtgtccttactctaagactgctgatggtttcgagactcacctcggagtcaaccacctgggccacttcctcctcacctacctgctcctggagcgactcaaggtgtctgcccctgcccgagtggttaacgtttcctccgtggctcaccacattggcaagattcccttccacgacctccagtccgagaagcgatactcccgaggttttgcttactgccactccaagctggccaacgttctctttacccgagagctggccaagcgactccagggaaccggcgtcaccacctacgccgttcaccccggtgtcgtccgatccgagctggtccgacactcctccctgctctgcctgctctggcgactcttctcccccttcgttaagaccgcccgagagggtgcccagacctccctgcactgcgccctggctgagggcctggagcccctgtctggcaagtacttctctgactgcaagcgaacctgggtgtctccccgagctcgaaacaacaagactgccgagcgactctggaacgtttcctgtgagcttctcggtattcgatgggagtag

SEQ ID NO: 6 - DNA, 900 bp, synthetic construct
  misc_feature 1..900: note = Yarrowia codon-optimized synthetic polynucleotide
  source 1..900: mol_type = other DNA
atgactccctccattcgaaagttctttgctggtggagtttgtactaccaaggtccagatccccggaaaggttgtggtcatcactggtgccaacactggcattggcaaggagactgcccgagagcttgctcgacgaggagcccgagtttacattgcctgtcgagatgtcctgaagggagagtctgctgcctctgagattcgagccgataccaagaactcccaggttctcgtgcgaaagctggacctctctgacaccaagtctatccgaacctttgctgagggcttccttgctgaggagaagaagctgcacattcttattaacaacgctggcgtgatgatgtgtccttactctaagactgtcgatggttttgagactcactttggtgtcaaccacctgggccacttccttctcacttaccttctccttggccgactgaaggagtctgctcccgcccgagtgattaacctctcttccgtggctcaccttggtggcaagatccgatttcacgaccttcagtccaagaagcgatactgctctggtttcgcttactctcactccaagctggccaacgtccttttcacccgagagctggccaagcgactccagggaaccggagtcaccgcttacgtggttcaccctggttgcgtcctgtctgagatcacccgacactccttcctgatgtgccttctctggcgactcttctcccccttcttcaagtccccttggcagggagcccagacctccctgcactgcgccctggaggagggcctggagcccctgtccggaaagtacttctctgactgcaagcgaacctgggtttctccccgagctcgaaacaagaagactgccgagcgactgtggaacgtttcctgtgagcttctcggaatccagtgggagtaa

SEQ ID NO: 7 - DNA, 1887 bp, synthetic construct
  misc_feature 1..1887: note = Yarrowia codon-optimized synthetic polynucleotide
  source 1..1887: mol_type = other DNA
atgctcactcccgccgctgagaacccccttcgagagcagggactccctgccccctctcccaccggatacaacaacgttcccgctttcaacaagcctgttgagcttaccattgagggcactattcctgagtgggttaacggtgtcatgtaccgagctggttctggccgatacaaccttctccttgagaacggcgataccttccacatcggacaccctttcgatggtctggctatgcttcaccgatttgagctttctggtgagactcagactgttcagtactcctcccgacacacctcccacggagttgagcgacgaatccgagagaaggaccctacccttctcacctttggtcctgacccctgtaagaccatttttggccgaatccagtccgtttaccaccacatctccaagttcggcgctaacgctcagattcaggagggcgaccccgagttcgatatggttaacgttaccattacccctaacttcccccttggtgagcgactggaggccgagactggtgttaagcgaggcgatgctcttgttgtcaagcgagatgctaacacccttcagctcgttgataacaagacccttaagcctatcaagatgttcacttacggtcacgttaacgataagctccagggtcagctttgcgcttctcaccaccagtacgatgaggagactgacgagtacgttaacttcaccgttcgactcggtcctattccctcttttcagtcttacacccttggtccttaccttcccactccccctggctctaaggagaagatgcctgcccctcaggttcgacttcacgagcctatttaccgacaccttggtgcctggcgaacccttgagcctctcaagcctgcttacattcactccttctccatgactaagaactacattatcgttcctaacttcccttactactactctttcggaggcatgtccgccctttactactcttgtgcttaccagactttctactgggatgagactcgacccactctctttcacgttgttgaccgaaacactggccgacacgttgctacttacgatgctgacccttgcttttctttccactctgctaacgcttgggatgaggaggtcgatctccctggtggtggtaaggagcgagtgatttacatggactactgtgtttacgagaacactgacattgtcgatgcttctttcgatctcggtaagactcccactggttttgacgcttccaaggtcgagcctgctcgattcaagatcaagcgacacaccgatgacaagaaggataactccatttctccttcccagcttcgacgataccgacttggtaacgttcctgtctcctctaacgctcctgagacttcccgatggtcccctaagggaattaccggcctcctttccggcatttttgactttaacaagcgacgagtggcttcttacactgttcttggctccgatattgagcttccccgattcaactctaacttcaacctgcgaaagtaccgatacgtgtggggtgtttgtgagtctaagcacgctccttcttacgcttctggtgccgttgttaacggtcttattaagctcgatctcgataagcctaccctttgcaagaacactgaggagggttcctctgctaagatttgggatgagcccggctgttcttgttccgagcccatttttgtcgcccaccctgagcagcgagctgaggatgacggtgttcttatttctactgtcaacactaccacccctgacggaaaggagtcttgctttctccttatcgttgatgctgctactatggttgaggttggccgaaccactctcggtgctttcactgccatgactatccacggctcttttgtcgataccaacggaaagggtgttgctgttaactaa